(19) United States
(12) Patent Application Publication
(10) Pub. No.: US 2005/0123492 A1
(43) Pub. Date: Jun. 9, 2005

(54) Composition for skin whitening containing lysophosphatidylethanolamine as an active ingredient

(75) Inventors: Guk Hoon Chung, Yongin-city (KR); Young Sei Park, Soowon-city (KR); Wang Keun Choi, Soowon-city (KR); Chang Seo Park, Gwacheon-si (KR)

Correspondence Address: MERCHANT & GOULD PC, P.O. BOX 2903, MINNEAPOLIS, MN, 55402-0903, US

(73) Assignee: Doosan Corporation, Seoul (KR)

(21) Appl. No.: 10/503,200

(22) PCT Filed: Jan. 29, 2003
(86) PCT No.: PCT/KR03/00212

(30) Foreign Application Priority Data
Feb. 2, 2002 (KR) 10 2992 0006064

(51) Int. Cl. A61K 7/135; A61K 7/06

(52) U.S. Cl. 424/62; 424/74

(57) ABSTRACT

Lysophosphatidylethanolamine has skin whitening ability and thus a composition containing lysophosphatidylethanolamine as an active ingredient can be used to relieve hyperpigmentation or discoloration. The composition containing lysophosphatidylethanolamine can also induce moisturizing effect on skin, is very safe, and very stable.

### FIELD OF THE INVENTION

The present invention relates to a skin whitening composition. More particularly, the present invention relates to a skin whitening composition which comprises lysophosphatidylethanolamine, has remarkably improved whitening effect in addition to moisturizing effect, and is stable and safe.

### BACKGROUND OF THE INVENTION

Problems involved in skin pigmentation such as hyper-pigmentation, discoloration and freckle are caused by melanin produced excessively in epidermal chromatocytes by hormone imbalance or UV stimulation and then hyper-pigmented in epidermis. Conventionally, in order to prevent pigmentation or freckle, substances repressing the production of melanin such as L-ascorbic acid were administered in massive doses, and ointment, cream, lotion, etc. containing morus bark extract, kojic acid or a derivative thereof, hydroquinone, arbutin, etc. were applied locally.

However, most of them had problems in view of stability, safety and smell, or in other respects. Furthermore, the effects thereof were not enough to satisfy consumers or patients.

### DETAILED DESCRIPTION OF THE INVENTION

Therefore, the present invention has been made in view of the above problems. The object of the present invention is to provide a skin whitening composition comprising lysophosphatidylethanolamine (LPE) as an active ingredient for applying on skins, which deletes or relieve hyper-pigmentation or discoloration, has excellent stability and safety as well as additional moisturizing effect.

In order to accomplish the above object, the present invention is directed to a composition for skin whitening comprising lysophosphatidylethanolamine as an active ingredient.

Preferably, the lysophosphatidylethanolamine is one selected from the group consisting of lysophosphatidylethanolamine derived from animals, lysophosphatidylethanolamine derived from plants and lysophosphatidylethanolamine derived from phosphatidylethanolamine.

It is preferable that the lysophosphatidylethanolamine is contained in the content of 0.001 to 20.0% by weight based on the total weight of the composition.

Preferably, the composition further comprises one or more selected from the group consisting of morus bark extract, kojic acid or a derivative thereof, L-ascorbic acid or a derivative thereof and hydroquinone or a derivative thereof as active gradients.

Also, preferably, the hydroquinone derivative is β-D-glucose(arbutin).

Preferably, the composition further comprises one or more selected from the group consisting of UV-blocker and UV-absorber.

Preferably, the active ingredients other than lysophosphatidylethanolamine are contained in the content of 0.0001 to 20.0% by weight based on the total weight of the composition.

### BEST MODE FOR CARRYING OUT THE INVENTION

These inventors discovered that LPE exhibits excellent whitening effect when applied on skin in the form of a cosmetic composition.

Also, there inventors discovered that skin whitening substances such as L-ascorbic acid or a derivative thereof, morus bark extract, kojic acid or a derivative thereof, hydroquinone or a derivative thereof, arbutin and apple extract may be combined with LPE for producing skin whitening compositions.

LPE used herein exists naturally in the cells of plants or animals. In particular, egg yolk or brain cells contain a lot of LPE. LPE may be induced from phosphatidylethanolamine, a sort of phospholipid which exists in cell membranes. Phosphatidylethanolamine is contained in plenty in egg yolk or soy bean lecithin. Phosphatidylethanolamine contains two fatty acids per molecule. In vivo, LPE may be produced by deleting one fatty acid of sn-2 position from phosphatidylethanolamine with Phospholipase A2 of a phospholipid hydrolase.

L-ascorbic acid used herein exhibits an inhibitory action on the tyrosinase reaction, the controlling step of the melanin action, due to the strong reducing action and exhibits a reducing action on melanin. Further, as derivatives of L-ascorbic acid, for example, L-ascorbyl monoalkyl esters such as L-ascorbyl monostearate, L-ascorbyl monopalmitate, and L-ascorbyl monooleate; L-ascorbyl monoester derivatives such as L-ascorbyl monophosphate esters and L-ascorbyl-2-sulfate esters: dialkyl esters such as L-ascorbyl distearate, L-ascorbyl dipalmitate, and L-ascorbyl dioleate; L-ascorbyl diesters such as L-ascorbyl diphosphate esters; trialkyl esters such as L-ascorbyl tristearate, L-ascorbyl tripalmitate, and L-ascorbyl triooleate; ascorbyl trimesters such as L-ascorbyl triphosphate esters, etc. may be mentioned.

Particularly preferable as the L-ascorbic acid and its derivatives are L-ascorbic acid, L-ascorbyl phosphate esters, L-ascorbyl-2-sulfate esters, or their salts.

Morus bark extract used herein comprises dried root bark derived from Moraceae plants, for example, Morus alba Linne, M. bombicis Kodzumi, M. alba L. var. romana Loddiges and M. lhou Koidzumi.

As the kojic acid derivatives usable in the present invention, for example a kojic acid ester such as a kojic acid alkyl ester or a kojic acid ether such as a kojic acid alkyl ether may be mentioned.

As the glycoside of the hydroquinone usable in the present invention, for example, hexose glycosides such as hydroquinone α-D-glucose, hydroquinone β-D-glucose, hydroquinone α-L-glucose, hydroquinone β-L-glucose, hydroquinone α-D-galactose, hydroquinone β-D-galactose, hydroquinone α-L-galactose, or hydroquinone β-L-galactose; pentose glycosides such as hydroquinone α-D-ripose, hydroquinone β-D-ripose, hydroquinone α-L-ripose, hydroquinone β-L-ripose, hydroquinone α-D-arabinose, hydroquinone β-D-arabinose, hydroquinone α-L-arabinose, and hydroquinone β-L-arabinose; aminosugar glycosides such as hydroquinone α-D-glucosamine, hydroquinone β-D-glucosamine, hydroquinone α-L-glucosamine, hydroquinone β-L-glucosamine, hydroquinone α-D-galactosamine, hydroquinone β-D-galactosamine, hydroquinone α-L-galactosamine, and hydroquinone β-L-galactosamine; and uronic acid glycosides such as hydroquinone α-D-gluconic acid, hydroquinone β-D-gluconic acid, hydroquinone α-L-gluconic acid, hydroquinone β-L-gluconic acid, hydroquinone α-D-galucturonic acid, hydroquinone β-D-galucturonic acid, hydroquinone α-L-galucturonic acid, hydroquinone β-L-galucturonic acid, etc.

Further, as their derivatives, an ester such as an acetylate, an ether such as a methylate, etc. may be mentioned, but judging from the whitening effect, the ease of acquisition, the shelf life, etc., use of hydroquinone β-D-glucose (general name: arbutin, hereinafter called “arbutin”) is preferable.

The content of the one or more types of components selected from the group consisting of L-ascorbic acid and its derivatives, morus bark extracts, kojic acid and its derivatives, hydroquinone and its derivatives, arbutin and apple extracts is not particularly limited, but in general is 0.0001% to 30.0% by weight based upon the total weight of the composition, preferably 0.0001 to 20.0% by weight.

Further, a UV protector may be further formulated into the composition of the present invention so as to further improve the whitening effect.

The UV protector used in the present invention includes both a “UV absorber” for absorbing UV rays physiochemically and a “UV blocker” for scattering and reflecting UV rays by physical means. These UV absorbers and UV blockers may be used alone or in any combinations thereof.

As the UV absorber, benzoate-based UV absorbers such as para-aminobenzonic acid (hereinafter referred to as “PABA”), PABA monoglycerin ester, N,N-dipropoxy PABA-ethyl ester, N,N-diethoxy PABA ethyl ester, N,N-dimethyl PABA-ethyl ester, N,N-dimethtl PABA-butyl ester, and N,N-dimethyl PABA-amyl ester; anthranilic acid-based UV absorbers such as homomenthyl-N-acethyl anthranilate etc.; salicylate-based UV absorbers such as amylsalicylate, menthylsalicylate, homomenthylsalicylate, octylsalicylate, phenylsalicylate, benzylsalicylate, and p-isopropanol phenyl-salicylate; cinnamate-based UV absorbers such as octylcinnamate, ethyl-4-isopropylcinnamate, methyl-2,5-diisopropylcinnamate, ethyl-2,4-diisopropylcinnamate, methyl-2,4-diisopropylcinnamate, methyl-2,4-diisopropylcinnamate, propyl-p-methoxycinnamate, isopropyl-p-methoxycinnamate, isoamyl-p-methoxycinnamate, octyl-p-methoxycinnamate (2-ethylhexyl-p-methoxycinnamate), 2-ethoxyethyl-p-methoxycinnamate, cyclohexyl-p-methoxycinnamate, ethyl-α-cyano-β-phenylcinnamate, 2-ethylhexyl-α-cyano-β-phenylcinnamate, glycerylmono-2-ethylhexanoyl-diparamethoxycinnamate, and 3,4,5-trimethyl-cinnamate 3-methyl-4-[methylbis(trimethylsiloxy)silyl]butyl; benzophenone-based UV absorbers such as 2,4-dihydroxybenzophenone, 2,2′-dihydroxy-4-methoxybenzophenone, 2,2′-dihydroxy-4,4′-dimethoxybenzophenone, 2,2,4,4′-tetrahydroxybnzophenone, 2-hydroxy-4-methoxybenzophenone, 2-hydroxy-4-methoxy-4′-methylbenzophenone, 2-hydroxy-4-methoxybenzophenone-5-sulfonic acid, 4-phenylbenzophenone, 2-ethylhexyl-4′-phenyl-benzophenone 2-carboxylate, 2-hydroxy-4-n-octoxyenzophenone, and 4-hydroxy-3-carboxybenzophenone; 3-(4′-methylbenzylidene)d, 1-comphor, 3-benzylidene d, 1-camphor, urocanic acid, urocanate ethyl esters, 2-phenyl-5-methylbenzoxanole, 2,2-hydroxy-5-methylphenylbenzotriazole, 2-(2′-hydroxy-5′-t-octylphenyl)benzotriazole, 2-(2′-hydroxy-5′-methylphenylbenzotriazole, dibenzalazine, dianisoylmethane, 4-tert-butyl-4′-methoxydibenzoylmethane, 5-(3,3-dimethyl-2-norbornylidene)-3-pentan 2-one, etc. may be mentioned. Particularly preferably 4-tert-butyl-4′-methoxydibenzoylmethane, octyl-p-methoxycinnamate, 2-hydroxy-4-methoxybenzophenone, 2-hyroxy-4-methoxybenzophenone-5-sulfonate, etc. may be mentioned.

Further, as the UV blockers, titanium dioxide (TiO₂), talc (MgSiO₂), carmine (FeO₂), bentonite, kaolin, zincoxide (ZnO), etc. may be mentioned.

When blending in such a UV protector, the amount blended normally is preferably 0.0001 to 30.0% by weight of the total weight of the external skin treatment composition, more preferably 0.0001 to 20.0% by weight.

The composition of the present invention may have suitably formulated therein, in addition to the above essential components, other components normally used for external skin treatment compositions such as cosmetics or pharmaceuticals, for example, oils, moisturizers, anti-oxidants, surfactants, preservatives, moisture retention agents, perfumes, water, alcohols, thickeners, etc., if desired.

The carrier of the composition according to the present invention may be of any type. For example, it may be made solubilized type such as cosmetic water, emulsion, cream, any other type such as ointment, dispersion.

### EXAMPLES

The present invention will now be explained in further detail with reference to Examples, but the technical scope of the present invention is by no means limited to these Examples.

These inventors produced following compositions in order to compare them with the examples according to the present invention.

TABLE 1

| | Com- | Com- | Com- | Com- | Com- |
|---|---|---|---|---|---|
| | parative | parative | parative | parative | parative |
| Components (% by | Ex- | Ex- | Ex- | Ex- | Ex- |
| weight) | ample 1 | ample 2 | ample 3 | ample 4 | ample 5 |
| Stearic acid | 1.5 | 1.5 | 1.5 | 1.5 | 1.5 |
| Squalane | 15 | 15 | 15 | 15 | 15 |
| 2-Octydodecyl | 2 | 2 | 2 | 2 | 2 |
| alcohol | | | | | |
| Polyoxyethylene(25 mol) | 1.2 | 1.2 | 1.2 | 1.2 | 1.2 |
| cetyl alcohol | | | | | |
| ether | | | | | |
| Glyceryl | 2.5 | 2.5 | 2.5 | 2.5 | 2.5 |
| monostearate ester | | | | | |
| Preservative and | q.s. | q.s. | q.s. | q.s. | q.s. |
| antioxidant | | | | | |
| Stearyl alcohol | 2.5 | 2.5 | 2.5 | 2.5 | 2.5 |
| Sorbitan | 0.6 | 0.6 | 0.6 | 0.6 | 0.6 |
| sesquioleate | | | | | |
| LPE | — | — | — | — | — |
| Morus bark extract | — | 1 | — | — | — |
| Kojic acid | — | — | 1 | — | — |
| L-ascorbic acid | — | — | — | 1 | — |
| Arbutin | — | — | — | — | 1 |
| Glycerin | 5 | 5 | 5 | 5 | 5 |
| Ion exchanged | To 100 | To 100 | To 100 | To 100 | To 100 |
| water | | | | | |

The process for producing the compositions according to the above Comparative Examples 1 to 5 is as follows:

An aqueous phase was heated to 70° C. (aqueous phase). The remainder of the components were mixed, then heated to melt and then maintained to 70° C. (Oil phase). Then, the oil phase was added to the aqueous phase to preliminarily emulsify, the mixture was emulsified with a homogenizer, and then cooled to 40° C. while stirring well. And, then preservative, morus bark extract, L-ascorbic acid, etc., which are sensitive to heat, were added to the emulsion, the mixture was emulsified with a homogenizer, and then cooled to 30° C.

Also, these inventors produced the compositions according to the present invention as follows.

TABLE 2

| Components | | Ex- | Ex- | Ex- | Ex- |
|---|---|---|---|---|---|
| (% by weight) | Example 1 | ample 2 | ample 3 | ample 4 | ample 5 |
| Stearic acid | 1.5 | 1.5 | 1.5 | 1.5 | 1.5 |
| Squalane | 15 | 15 | 15 | 15 | 15 |
| 2-Octydodecyl alcohol | 2 | 2 | 2 | 2 | 2 |
| Polyoxyethylene(25 | 1.2 | 1.2 | 1.2 | 1.2 | 1.2 |
| mol)cetyl alcohol ether | | | | | |
| Glyceryl monostearate | 2.5 | 2.5 | 2.5 | 2.5 | 2.5 |
| ester | | | | | |
| Preservative and | q.s. | q.s. | q.s. | q.s. | q.s. |
| antioxidant | | | | | |
| Stearyl alcohol | 2.5 | 2.5 | 2.5 | 2.5 | 2.5 |
| Sorbitan sesquioleate | 0.6 | 0.6 | 0.6 | 0.6 | 0.6 |
| LPE | 1 | 1 | 1 | 1 | 1 |
| Morus bark extract | 1 | 1 | 1 | 1 | — |
| Kojic acid | 1 | 1 | 1 | — | — |
| L-ascorbic acid | 1 | 1 | — | — | — |
| Arbutin | 1 | — | — | — | — |
| Glycerin | 5 | 5 | 5 | 5 | 5 |
| Ion exchanged water | To 100 | To 100 | To 100 | To 100 | To 100 |

The compositions according to the Examples 1 to 5 described in the above Table 2 were produced by the following method:

An aqueous phase was heated to 70° C. (aqueous phase). The remainder of the components were mixed, then heated to melt and then maintained to 70° C. (Oil phase). Then, the oil phase was added to the aqueous phase to preliminarily emulsify, the mixture was emulsified with a homogenizer, and then cooled to 40° C. while stirring well. And, then preservative, morus bark extract, L-ascorbic acid, etc., which are sensitive to heat, were added to the emulsion, the mixture was emulsified with a homogenizer, and then cooled to 30° C.

### Experimental Example 1

### Whitening Effect

These inventors tested the compositions according the above Examples 1 to 5 and the Comparative Examples 1 to 5 as follows in order to demonstrate the skin whitening effect of the present invention.

(Test Method)

A group of 20 test subjects suffering from dark complexion, pigmentation, freckles, etc. were asked to coat sample lotions of Examples 1 to 8 and Comparative Examples 1 to 9 every day on the face in the morning and evening for three months. After three months, the whitening effect was investigated. The extent of the dark complexion, pigmentation, and freckles before and after the start was evaluated in seven ranks:

(Evaluation Criteria)
1: No dark complexion, pigmentation, and freckles.
2: Minor degree of dark complexion, pigmentation, and freckles.
3: Light degree of dark complexion, pigmentation, and freckles.
4: Medium light degree of dark complexion, pigmentation, and freckles.
5: Medium degree of dark complexion, pigmentation, and freckles.
6: Medium high degree of dark complexion, pigmentation, and freckles.
7: High degree of dark complexion, pigmentation, and freckles.

(Evaluation)
++: At least 80% of test subjects reported improvement of at least 2 ranks (valid ratio)
+: At least 50% to less than 80% of test subjects reported improvement of at least 2 ranks (valid ratio)
±: At least 30% to less than 50% of test subjects reported improvement of at least 2 ranks (valid ratio)
−: Less than 30% of test subjects reported improvement of at least 2 ranks (valid ratio)

The results of the whitening effects of the Comparative Examples 1 to 5 and Examples 1 to 5 tested by the above method are as follows:

TABLE 3

| | Whitening Effect |
|---|---|
| Comparative Example 1 | − |
| Comparative Example 2 | − |
| Comparative Example 3 | ± |
| Comparative Example 4 | ± |
| Comparative Example 5 | ± |
| Example 1 | ++ |
| Example 2 | ++ |
| Example 3 | ++ |
| Example 4 | ++ |
| Example 5 | + |

As shown in the above Table 1, the compositions for skin whitening containing LPE according to the present invention (Examples 1 to 5) exhibit more improved whitening effect than those of Comparative Examples 1 to 5. Also, the composition comprising morus bark extract, kojic acid or L-ascorbic acid in addition to LPE (Examples 1 to 4) exhibit more superior whitening effect than those containing only LPE (Example 5).

### Experimental Example 2

### Moisture Retention Effect

Twenty healthy subjects subjected to different amounts of UV irradiation in advance and measured for minimum erythema dose (MED) were irradiated at their stomach portions to 1.44 MED using two lamps, and FL40SE lamp and a BLB lamp (made by Toshiba). 2×2 cm portions irradiated by UV rays were determined as portions for coating the samples of Examples 1 to 8 and Comparative Examples 1 to 9, portions for coating samples of the same formulations as the Examples and Comparative Examples but without the medicines, and portions not coated with anything (control) and the treatment continued for one week. After the one week, the skin conductance was measured in a constant temperature, constant humidity room (using SKICON-200 made by IBS Co.) and the corneal moisture content was found.

The values of the corneal moisture content of the portions coated with the samples of the Examples and Comparative Examples indexed to the corneal moisture content of the portions coated with samples not containing any medicines as “1” and the average values of the values of the corneal content of the portions coated with the samples of the Examples indexed to the corneal content of the control as “1” are shown in Table 4 and Table 5.

TABLE 4

| | Moisture Retention Effect | |
|---|---|---|
| | Based on General Cream | Based on Control |
| Comparative Example 1 | 1 | 1.5 |
| Comparative Example 2 | 1.2 | 1.8 |
| Comparative Example 3 | 1.1 | 1.3 |
| Comparative Example 4 | 1 | 1.4 |
| Comparative Example 5 | 1.3 | 1.6 |
| Example 1 | 1.2 | 2.4 |
| Example 2 | 1.3 | 2.5 |
| Example 3 | 1.1 | 2.4 |
| Example 4 | 1.2 | 2.3 |
| Example 5 | 1.2 | 2.3 |

As clear from the results of Table 4 and Table 5, it was observed that all Comparative Examples and Examples exhibit more excellent moisture retention effects than Control that no treatment was carried out. Also, the Comparative Examples and Examples containing LPE, morus bark extract, kojic acid or L-ascorbic acid exhibit more superior moisture retention effects than the general cream according to Comparative Example 1, which does not contain any one of them. Especially, the compositions of Comparative Examples 2 to 5 not having LPE exhibit no improved moisturizing effect or just a little improvement, while the compositions of all Examples containing LPE exhibit considerably improved moisture retention effects. Therefore, it was demonstrated that the compositions containing LPE as an active ingredient have improved moisture retention effect in addition to skin whitening effect.

These inventors suggested formulation examples of the composition according to the present invention as follows.

### Formulation Example 1

### Nutrition Cream

| | | |
|---|---|---|
| Stearyl alcohol | 6.0 | wt % |
| Stearic acid | 1.0 | |
| Hydrogenated lanolin | 2.0 | |
| LPE | 1.5 | |
| Squalane | 5.0 | |
| 2-Octydodecyl alcohol | 6.0 | |
| Polyoxyethylene(25 mol)cetyl alcohol ether | 1.2 | |
| Glyceryl monostearate ester | 2.0 | |
| Glycerin | 5.0 | |
| Perfume | q.s. | |
| Preservative and antioxidant | q.s. | |
| Ion exchanged water | Bal. | |

The nutrition cream was produced by the following method: an aqueous phase was heated to 70° C. (aqueous phase). The remainder of the components were mixed, then heated to melt and then maintained to 70° C. (Oil phase). Then, the oil phase was added to the aqueous phase to preliminarily emulsify, the mixture was emulsified with a homogenizer, and then cooled to 40° C. while stirring well. And, then preservative, morus bark extract, L-ascorbic acid, etc., which are sensitive to heat, were added to the emulsion, the mixture was emulsified with a homogenizer, and then cooled to 30° C.

### Formulation Example 2

### Cleansing Cream

| | | |
|---|---|---|
| Solid paraffin | 1.0 | wt % |
| Beeswax | 1.5 | |
| Liquid paraffin | 41.0 | |
| Vaseline | 3.0 | |
| Glyceryl monostearate ester | 2.5 | |
| Polyoxyethylene(20 mol)sorbitan | 2.0 | |
| monolaurate ester | | |
| Cetostearyl alcohol | 1.5 | |
| LPE | 1.0 | |
| Carboxy vinyl polymer | 0.2 | |
| Ion exchanged water | Bal. | |
| Triethylamine | 0.15 | |
| Perfume | q.s. | |
| Preservative and antioxidant | q.s. | |

The cleansing cream according to the Formulation Example 2 was produced by the method as follows: an aqueous phase was heated to 70° C. (aqueous phase). The remainder of the components were mixed, then heated to melt and then maintained to 70° C. (Oil phase). Then, the oil phase was added to the aqueous phase to preliminarily emulsify, the mixture was emulsified with a homogenizer, and then cooled to 40° C. while stirring well. And, then preservative, morus bark extract, L-ascorbic acid, etc., which are sensitive to heat, were added to the emulsion, the mixture was emulsified with a homogenizer, and then cooled to 30° C.

### Formulation Example 3

### Emulsion

| | | |
|---|---|---|
| Polysorbate 60 | 1.0 | wt % |
| Octyl-p-methoxycinnamate | 3.5 | |
| Silicone KF96 (20 cs) | 2.0 | |
| (made by Shin-Etsu Chemical) | | |
| Liquid paraffin (medium viscosity) | 3.0 | |
| Squalane | 3.0 | |
| Glycerin | 5.0 | |
| Arbutin | 1.0 | |
| LPE | 1.0 | |
| Sorbitan sesquioleate | 0.3 | |
| Propylene glycol | 2.0 | |
| Ethanol | 10.0 | |
| Carboxy vinyl polymer | 0.3. | |
| KOH | q.s. | |
| Preservatives | q.s. | |
| Ion exchanged water | Bal. | |

The emulsion according to the Formulation Example 3 was produced by the method as follows: an aqueous phase was heated to 70° C. (aqueous phase). The remainder of the components were mixed, then heated to melt and then maintained to 70° C. (Oil phase). Then, the oil phase was added to the aqueous phase to preliminarily emulsify, the mixture was emulsified with a homogenizer, and then cooled to 40° C. while stirring well. And, then preservative, morus bark extract, L-ascorbic acid, etc., which are sensitive to heat, were added to the emulsion, the mixture was emulsified with a homogenizer, and then cooled to 30° C.

### Formulation Example 4

### Pack

| | | |
|---|---|---|
| Purified water | To 100 | wt % |
| Sodium carboxy methyl cellulose | 0.05 | |
| 1,3-buthylene glycol | 1.5 | |
| Ethanol | 12.0 | |
| Nonylphenylether | 0.4 | |
| Preservative | q.s. | |
| LPE | 0.2 | |
| Polyvinyl alcohol | 11.0 | |
| Perfume | q.s. | |

The Pack according the above Formulation Example 4 was produced by the method as follows: alcohol-soluble components were dipped in alcohol. Then, an aqueous phase was heated to 70° C. and the alcohol phase was added to the aqueous phase slowly to mix them. After confirming the complete dissolving, the mixture was cooled to 30° C.

### Formulation Example 5

### Softener

| | | |
|---|---|---|
| Glycerin | 5.0 | wt % |
| Propylene glycol | 3.0 | |
| Carboxyvinyl polymer | 0.1 | |
| Nonylphenyl ether | 0.3 | |
| Ethanol | 10.0 | |
| Triethylamine | 0.1 | |
| LPE | 0.5 | |
| Preservatives | q.s. | |
| Purified water | Bal. | |

The softener according to the above Formulation Example 5 was produced by the method as follows: alcohol-soluble portions were added to alcohol portions to dissolve. An aqueous phase was added to the purified water and then the complete solution was confirmed. Then, the alcohol portions were added to the aqueous phase slowly to mix. Especially, triethylamine was added at last.

The cosmetic composition or external treatment composition according to the present invention containing LPE as an active ingredient is able to delete or relive pigmentation or freckles on skin. In addition to such skin whitening effect, the composition has superior moisture retention effect, stability and safety.

1. A composition for skin whitening comprising lysophosphatidylethanolamine as an active ingredient.

2. The composition according to claim 1, wherein the lysophosphatidylethanolamine is one selected from the group consisting of lysophosphatidylethanolamine derived from animals, lysophosphatidylethanolamine derived from plants and lysophosphatidylethanolamine derived from phosphatidylethanolamine.

3. The composition according to claim 1, wherein the lysophosphatidylethanolamine is contained in the content of 0.001 to 20.0% by weight based on the total weight of the composition.

4. The composition according to claim 1, further comprising one or more selected from the group consisting of morus bark extract, kojic acid or a derivative thereof, L-ascorbic acid or a derivative thereof and hydroquinone or a derivative thereof as active gradients.

5. The composition according to claim 4, wherein the hydroquinone derivative is β-D-glucose(arbutin).

6. The composition according to claim 1, further comprising one or more selected from the group consisting of UV-blocker and UV-absorber.

7. The composition according to claim 4, the active ingredients other than lysophosphatidylethanolamine are contained in the content of 0.0001 to 20.0% by weight based on the total weight of the composition.

8. The composition according to claim 5, the active ingredients other than lysophosphatidylethanolamine are contained in the content of 0.0001 to 20.0% by weight based on the total weight of the composition.

9. The composition according to claim 6, the active ingredients other than lysophosphatidylethanolamine are contained in the content of 0.0001 to 20.0% by weight based on the total weight of the composition.
